(19) United States
(12) Patent Application Publication
(10) Pub. No.: US 2025/0236846 A1
(43) Pub. Date: Jul. 24, 2025

(54) High-Titer AAV2 Formulations Having Reduced Viral Aggregation

(71) Applicant: Oxford Biomedica (US) LLC, Bedford, MA (US)

(72) Inventors: Alex Meola, Bedford, MA (US); Kelly Walsh, Bedford, MA (US); Ashish Sharma, Bedford, MA (US); Zaid Junaid, Bedford, MA (US)

(21) Appl. No.: 18/986,164

(22) Filed: Dec. 18, 2024

Related U.S. Application Data
(60) Provisional application No. 63/612,097, filed on Dec. 19, 2023.

(51) Int. Cl. C12N 7/00

(52) U.S. Cl. CPC C12N 7/00; C12N 2750/14151

(57) ABSTRACT

Provided are compositions that provide reduced aggregation of AAV2 particles. Also provided are methods of making and using the compositions. The compositions provided by the present disclosure generally comprise an AAV2 and a buffer with an aggregation reduction agent, such as trehalose in a high concentration (i.e., greater than 5%).

### BACKGROUND

Gene therapy using adeno-associated viruses (AAV) has the potential to treat a wide variety of human disorders. One challenge with using AAVs for gene therapy is maintaining the colloidal stability of the viral particles during manufacture and storage of the final drug formulation. Although progress has been made regarding formulations that improve stability, certain serotypes, such as AAV2 are prone to aggregation, which hinders manufacturing, long-term storage, delivery, and efficacy of AAV2 formulations.

### SUMMARY

The present disclosure provides AAV2 compositions that can provide for reduced aggregation of AAV2 particles. Also provided herein are methods of making and using the compositions. The compositions provided by the present disclosure generally comprise AAV2 particles and a buffer comprising an additive (e.g., trehalose, glycerol, sucrose, and the like). The compositions described herein are particularly advantageous in that they have reduced aggregation of AAV2 particles when filtered and concentrated while maintaining high particle genome titers and maintaining AAV2 purity over time. Consequently, the compositions provided herein are particularly suitable for the long-term storage of AAV2 particles (e.g., AAV2 particles utilized as gene therapy particles).

Accordingly, provided herein is an AAV2 composition comprising AAV2 particles containing a transgene. The AAV2 product also contains a buffer comprising an aggregation-reducing additive (e.g., trehalose, sucrose, or glycerol) of greater than 5% w/v (e.g., 6-11% or 7-8%). The AAV2 composition has less particle aggregation than a control AAV2 composition in a buffer lacking the additive as measured, for example, by dynamic light scattering (DLS) or size exclusion chromatography. For example, aggregation can be detected at less than 100 nM or less than 50 nM for the Z-average diameter and less than 0.3 PDI with DLS after 3 diavolumes (DV) for diafiltration. For detection by size exclusion chromatography, the percentage of aggregated particles can be is less than 1% or less than 0.5%.

Optionally, the buffer of the AAV2 composition is neither a citrate buffer nor a potassium buffer. Optionally, the buffering agent of the buffer is histidine (e.g., at a concentration of 15-25 mM or 17-18 mM). Salt in the buffer of the AAV2 composition is optionally sodium chloride (e.g., at a concentration of 160-190 mM. The buffer optionally contains a surfactant, such as Poloxamer-188 (e.g., at a concentration of 0.0005-0.0015 (w/v %).

The AAV2 composition optionally has a pH of 7.3. The AAV2 composition can be either a chromatography product or an ultrafiltration/diafiltration product. By way of example, the chromatography product can be an anion-exchange chromatography (AEX) product, a hydrophobic interaction chromatograph (HIC) product, or a mixed mode chromatography product.

The AAV2 composition optionally comprises greater than 1×1013 viral genomes per milliliter (vg/ml), including, for example, 5-8×1013 vg/ml or 7-8×1013 vg/ml. The AAV2 composition is optionally stable (i.e., does not aggregate more than 10%) for at least one month at 2-8° C., for at least one year at 2-8° C.), or for at least two years at 2-8° C.).

Also provided herein is a method of producing an AAV2 product by filtering an isolated AAV2 composition using ultrafiltration/diafiltration (UFDF) to produce an AAV2 UFDF product, wherein the AAV2 UFDF product comprises AAV2 particles containing a transgene and a buffer comprising trehalose of greater than 5% w/v. The buffer comprising trehalose can be added before UFDF and optionally after diafiltration. Notably, the amount of aggregation is comparable before and after UFDF.

Also provided is an AAV2 product method made by the process. The product is suitable for administration to a cell (in vivo or in vitro).

### BRIEF DESCRIPTION OF DRAWINGS

FIG. 1 is a schematic showing an overview of the ultrafiltration diafiltration process used to produce the AAV2 ultrafiltration diafiltration (UFDF) product from the AAV2 anion exchange chromatography (AEX) product.

FIG. 2A-2B are bar graphs showing the level of aggregation of AAV2 particles during diafiltration as measured with dynamic light scattering (DLS). FIG. 2A shows the Z-average diameter and FIG. 2B shows the polydispersity index (PDI) at various diavolumes.

FIG. 3 is a schematic of an AAV2 ultrafiltration diafiltration process showing addition of a pre-formulation buffer containing 8% (W/v %) trehalose to the AEX product, resulting in a 7% final concentration of trehalose in the diluted AEX product. A volume of pre-formulation buffer equivalent to 1.7× the system volume was added after diafiltration and depolarization. This is referred to as the chase.

FIG. 4 is a plot of aggregate % for samples from different stages of the process shown in FIG. 3.

FIG. 5A-5B are bar graphs showing the level of aggregation of AAV2 particles with dilution and diafiltration in a buffer having 8% trehalose according to the method shown in FIG. 3. FIG. 5A shows the Z-average diameter and FIG. 5B shows the polydispersity index (PDI) at various diavolumes.

FIG. 6 shows an analysis of permeate flux and transmembrane pressure (TMP) during the concentration (ultrafiltration) and buffer exchange (diafiltration) steps according to the method shown in FIG. 3.

FIG. 7A-7B are reads of peak diameter for particles in AEX and UFDF products according to the method shown in FIG. 3. The AEX product and UFDF product showed comparable peak diameter as a function of mass distribution (FIG. 7A) and as a function of intensity (FIG. 7B), indicating the AAV2 particles were stable during diafiltration/ultrafiltration.

### DETAILED DESCRIPTION

In order to produce recombinant AAV2 particles for delivery to a subject, methods of production as described herein are designed to reduce aggregation of the AAV2 particles as compared to the levels of aggregation that result from methods currently in use. Aggregation of AAV2 particles may occur at multiple points in a manufacturing process (particularly during purification steps). Such aggregation can occur in response to stress (e.g., thermal or mechanical stress, such as shear stress). By reducing aggregation along the product stream, the final product is a stable composition with higher product efficacy and lower immunogenicity for a given dose of viral particles as compared to compositions made by other methods. Thus, also provided herein are novel compositions containing AAV2 particles. As used herein, a reduction in aggregation can be any reduction discernable with routine tests of aggregation (comparing, for example, a test composition and a control composition) and can include partial or complete elimination of aggregation.

### Methods of Producing an AAV2 Composition

Provided herein is a method of producing a composition containing AAV2 particles and the composition produced by the method. The composition comprises AAV2 particles, optionally comprising a transgene, and has reduced AAV2 aggregation as compared to a composition produced with a control method. The compositions produced by the methods described herein are optionally suitable for administration to a cell either in vivo or in vitro. Suitability for administration to a cell means that the viral particles of the composition can be delivered to one or more target cells so as to allow a transgene within the viral particles to be expressed by the target cell or cells.

To produce the composition, a vector system comprising a helper plasmid (including at least a portion of E4, at least a portion of E2a, and virus-associated (VA) nucleic acids of an adenovirus genome), a Rep gene, a Cap gene, and, optionally, a transgene are introduced into a host cell (i.e., a producer cell). The vector system can be a two, three, or four plasmid system. Any of the vectors or vector systems disclosed herein can be introduced into cells (using any techniques known in the art, e.g., transfection, electroporation, and the like) for propagation of the vectors and/or for expression of a protein(s) encoded by the vector.

As used herein, the term introducing (i.e., transfecting or transducing), in the context of introducing a nucleic acid sequence, for example, one or more vectors described herein, refers to the translocation of the nucleic acid sequence from outside a cell to inside the cell. In some cases, introducing refers to translocation of the nucleic acid from outside the cell to inside the nucleus of the cell. Various methods of such translocation are contemplated, including but not limited to, electroporation, contact with nanowires or nanotubes, receptor mediated internalization, translocation via cell penetrating peptides, liposome mediated translocation, and the like.

When the vector systems comprising two or more vectors, as described throughout, are introduced into the host cells, the vectors can be introduced at various molar ratios most appropriate for the vectors in use, for example an equal molar (1:1) ratio for a two-vector system, or a 1:1:1 ratio for a three-vector system. The total amount of nucleic acid that is introduced into the cell is generally from about 0.1 μg DNA/1×106 cells to 4.0 μg DNA/1×106 cells. For example, the total amount of nucleic acid that is transfected or transduced into the cell, including the first vector, the second vector, and optionally a third vector and/or fourth vector is 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4 μg DNA/1×106 cells.

The host cells can be expanded to an appropriate cell density prior to introducing one or more vectors to generate AAV2 producer cells. The host cell containing the vector system is incubated under culture conditions that promote AAV2 particle production. In some embodiments, the host cell is a mammalian cell, for example, a mammalian cell selected from the group consisting of a COS cell, a CHO cell, a BHK cell, an MDCK cell, an HEK293 cell, an HEK293T cell, a HeLa cells, an NS0 cell, a PER.C6 cell, a VERO cell, a CRL7O3O cell, an HsS78Bst cell, a HeLa cell, an NIH 3T3 cell, a HepG2 cell, an SP210 cell, an R1.1 cell, a B-W cell, an L-M cell, a BSC1 cell, a BSC40 cell, a YB/20 cell, and a BMT10 cell. Optionally, the host cell is a cell that can be grown in suspension culture, for example, an HEK293 cell, an HEK293T, or an HEK293F cell.

The AAV2 producer cells are lysed after the culture step, and the recombinant AAV2 (rAAV2) particles are harvested. The crude lysate is clarified, and AAV2 particles are captured using affinity chromatography. The resulting rAAV2 composition produced is referred to herein as the chromatography product, which is then purified. Optionally, the composition is concentrated prior to purification.

Methods for purifying an AAV2 composition are known in the art. As used herein, purify or purification refers to the elimination of at least a portion of a non-AAV2 particle product, e.g., removal of host cell proteins, media components, nucleic acids, and/or empty AAV2 particles from a cell culture, to obtain a purified AAV2 composition. For example, in some methods at least 20%, 30%, 40%, 50%, 60%, 70%, 80%, 90%, 91%, 92%, 93%, 94%, 95%, 96%, 97%, 98%, 99% 99.5% or 100% of one or more non-AAV2 particle products (including empty particles) can be removed or eliminated during purification. In some cases, purification comprises a clarification step for the removal of cells and cellular debris, e.g., using differential centrifugation, density centrifugation and/or filtration; and/or one or more downstream chromatography steps to separate the AAV2 composition from various impurities in the clarified cell culture feed. See, e.g., International Patent Application Publication No. WO 98/22588, which describes methods for the production and purification of adenoviral vectors. Chromatographic methods for purification of virus from a host cell lysate, are also set forth in U.S. Pat. Nos. 6,008,036, 6,586,226, 5,837,520, 6,261,823, 6,537,793, and International Patent Application Publication Nos. WO 00/50573, WO 02/44348 and WO 03/078592, the contents of the entirety of each of which are incorporated herein by this reference. Various chromatographic and non-chromatographic methods can be used, including affinity chromatography, anion exchange chromatography, ultracentrifugation, diafiltration, and other methods known in the art.

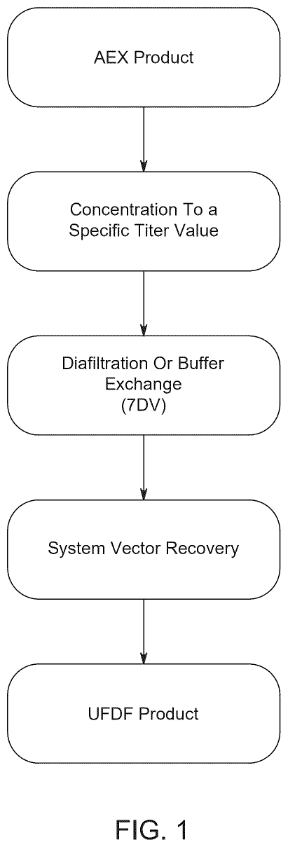

The purification process, as shown in FIG. 1, comprises a chromatography step (e.g., an anion exchange (AEX) chromatography step) to produce a chromatography product (e.g., an AEX product). The chromatography product is then concentrated with ultrafiltration (UF) to a selected titer value (e.g., a specific viral genome concentration) and subjected to diafiltration (DF) to produce a UFDF product. Optionally, the UFDF product is modified as necessary to achieve the final desired titer value. For example, the UFDF product can be diluted, concentrated, or combined with a composition of higher or lower titer value to achieve the final desired titer value. As described below in Example 1, particles of certain AAV serotypes, such as AAV2, aggregate during the purification steps and in particular during diafiltration and ultrafiltration. Thus, additional steps are needed create a high-titer AAV2 with aggregation reduced or eliminated.

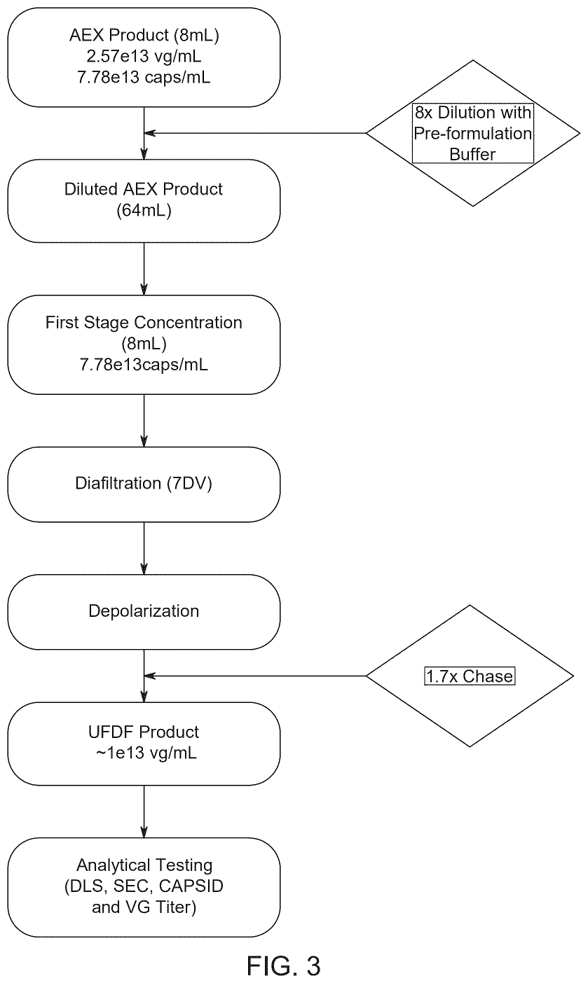

In the present method, as shown in FIG. 3, the chromatography product (e.g., the AEX product) is diluted with a pre-formulation buffer, which contains an additive at a concentration that reduces aggregation of the AAV2 particles. The diluted chromatography product is then subjected to ultrafiltration and diafiltration (e.g., for 1 diavolume (DV), 2 DV, 3 DV, 4 DV, 5 DV, 6 DV, 7DV, or more). Optionally, after diafiltration, additional buffer is added (referred to as the “chase”) to avoid an air liquid interface and to reduce the sheer rate in the ultrafiltration step. Optionally, the composition is depolarized prior to addition of the chase. The resulting UFDF product is optionally further tested to ensure product quality and/or concentration and to determine the level of aggregation (e.g., by DLS or size exclusion chromatography).

The buffer added to the chromatography product and/or to the product stream after diafiltration optionally comprises an additive at a concentration of greater than about 5% w/v, greater than about 6% or greater than about 7% (e.g., 6-11% or 7-8%). The additive is optionally an excluded additive, such as trehalose, sucrose, glycerol, or an included additive, such as monovalent sodium salts, urea, and guanidine. The buffer optionally is not a citrate buffer or a potassium buffer. The buffer optionally comprises histidine as a buffering agent. Histidine can be present in the buffer at a concentration of 15-25 mM, or more, specifically 17-18 mM. The buffer optionally contains a salt, such as sodium chloride. Sodium chloride is optionally present in the buffer at a concentration of 160-190 mM. The buffer optionally contains a surfactant, such as Poloxamer-188. The surfactant optionally is present in a concentration of about 0.000-0.0015% (w/v %). The buffer optionally has a pH of 6.5-7.5, for example, 7.0-7.5 or about 7.3.

The method of producing a composition comprising AAV2 particles minimizes aggregation across the product stream in a single run. In other words, the level of aggregation as described herein in a process stream is optionally comparable in both the diluted chromatography product and the UFDF product. By comparable is meant that the level of aggregation in the UFDF product as measured by DSL (Z-average or PDI) is within at least 15% (e.g., within 5%, 10%, or 15%) of the level of aggregation in the chromatography product.

### Compositions of Adeno-Associated Virus Type 2 (AAV2)

The present disclosure provides AAV2 compositions comprising AAV2 particles and a buffer. The buffer comprises an additive that reduces aggregation of AAV2 particles. The additive can be excluded additive (i.e., one that prevents aggregation by preventing motion of molecules, such as, trehalose, sucrose, or glycerol) or an included additive (i.e., an additive that interacts with the surface of AAV2 particles to solubilize potential aggregation sites on the particle surface, such as monovalent sodium salts, urea, and guanidine”). The buffer containing the additive that reduces aggregation of AAV2 particles can be added at various points in the production stream. Thus, the AAV2 composition containing the additive can be a chromatography product or a UFDF product. Adding the buffer to the chromatography product prior to UFDF reduces AAV particle aggregation that occurs during ultrafiltration and diafiltration. As described above, the buffer containing the additive that reduces aggregation can further comprise one or more excipients in addition to a buffering agent (e.g., histidine), for example, a salt (e.g., sodium chloride) and/or a surfactant (e.g., Poloxamer-188).

The compositions comprising the excluded or included additives and AAVs particles have reduced levels of particle aggregates as compared to comparable compositions lacking the additive or lacking a sufficient concentration of the additive. The AAV2 serotype is particularly prone to aggregation in traditional buffers, including buffers with low levels of the additive. For example, 3% (w/v) trehalose was insufficient to prevent aggregation at 3 DV, whereas 7-8% was sufficient to at least 7 DV.

Aggregation of AAV2 particles in a composition administered to a subject results in reduced efficacy and increase immunogenicity with a given dose of viral particles as compared to a composition lacking aggregated particles. The present disclosure provides compositions, including pharmaceutical compositions, comprising AAV2 particles and a buffer comprising an effective amount of an excluded or included additive selected for reducing aggregation. The AAV2 particles of the composition optionally contain a transgene. The compositions exhibit less AAV2 particle aggregation than a control AAV2 composition in a buffer lacking the excluded or included additive. The compositions are thus useful as compositions for use in a process for making a pharmaceutical composition (e.g., a chromatography product) or as a stable pharmaceutical composition for delivery of a transgene to a subject (e.g., a UFDF product).

### Adeno-Associated Virus Type 2 (AAV2)

Compositions disclosed herein comprise AAV2 particles, which optionally contain a transgene. The AAV2 particles are recombinant AAV2 (rAAV2) and are not naturally occurring, wild-type AAV2.

Generally, an AAV2 or rAAV2 genome comprises two open reading frames, Cap and Rep genes, flanked by two 145 base inverted terminal repeats (ITRs). These ITRs base pairs allow for synthesis of the complementary DNA strand. Rep and Cap are translated to produce multiple distinct proteins (Rep78, Rep68, Rep52, Rep40-required for the AAV life cycle; VP1, VP2, VP3-capsid proteins). In certain embodiments, the rAAV2 genome also comprises a transgene.

In certain embodiments, the transgene comprises one or more sequences encoding an RNA molecule. Suitable RNA molecules include, without limitation, miRNA, shRNA, siRNA, antisense RNA, gRNA, antagomirs, miRNA sponges, RNA aptazymes, RNA aptamers, mRNA, lncRNAs, ribozymes, and synthetic RNAs known in the art.

In certain embodiments, the transgene encodes one or more polypeptides or one or more fragments thereof. Such transgenes can comprise the complete coding sequence of a polypeptide, or only a fragment of a coding sequence of a polypeptide. In certain embodiments, the transgene encodes a polypeptide that is useful to treat a disease or disorder in a subject. Suitable polypeptides include, without limitation, β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-α), transforming growth factor beta (TGF-β), and the like; soluble receptors, such as soluble TNF-α receptors, soluble interleukin receptors (e.g., soluble IL-1 receptors and soluble type II IL-1 receptors), soluble γ/Δ T cell receptors, ligand-binding fragments of a soluble receptor, and the like; enzymes, such as α-glucosidase, imiglucerase, β-glucocerebrosidase, and alglucerase; enzyme activators, such as tissue plasminogen activator; chemokines, such as IP-10, monokine induced by interferon-gamma (Mig), Groα/IL-8, RANTES, MIP-1a, MIP-1β, MCP-1, PF-4, and the like; angiogenic agents, such as vascular endothelial growth factors (VEGFs, e.g., VEGF121, VEGF165, VEGF-C, VEGF-2), glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastrin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); tissue factors; macrophage activating factors; tumor necrosis factor (TNF); neutrophil chemotactic factor (NCF); tissue inhibitors of metalloproteinases; vasoactive intestinal peptide; angiogenin; angiotrofin; fibrin; hirudin; IL-1 receptor antagonists; ciliary neurotrophic factor (CNTF); brain-derived neurotrophic factor (BDNF); neurotrophins 3 and 4/5 (NT-3 and -4/5); glial cell derived neurotrophic factor (GDNF); aromatic amino acid decarboxylase (AADC); Factor VIII, Factor IX, Factor X; dystrophin or mini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase, glucose transporter, aldolase A, β-enolase, glycogen synthase; lysosomal enzymes, such as iduronate-2-sulfatase (I2S), and arylsulfatase A; and mitochondrial proteins, such as frataxin.

In certain embodiments, the transgene encodes a protein that may be defective in one or more lysosomal storage diseases. Suitable proteins include, without limitation, α-sialidase, cathepsin A, α-mannosidase, β-mannosidase, glycosylasparaginase, α-fucosidase, α-N-acetylglucosaminidase, β-galactosidase, β-hexosaminidase α-subunit, β-hexosaminidase β-subunit, GM2 activator protein, glucocerebrosidase, Saposin C, Arylsulfatase A, Saposin B, formyl-glycine generating enzyme, β-galactosylceramidase, α-galactosidase A, iduronate sulfatase, α-iduronidase, heparan N-sulfatase, acetyl-CoA transferase, N-acetyl glucosaminidase, β-glucuronidase, N-acetyl glucosamine 6-sulfatase, N-acetylgalactosamine 4-sulfatase, galactose 6-sulfatase, hyaluronidase, α-glucosidase, acid sphingomyelinase, acid ceramidase, acid lipase, cathepsin K, tripeptidyl peptidase, palmitoyl-protein thioesterase, cystinosin, sialin, UDP-N-acetylglucosamine, phosphotransferase γ-subunit, mucolipin-1, LAMP-2, NPC1, CLN 3, CLN 6, CLN 8, LYST, MYOV, RAB27A, melanophilin, and AP3 β-subunit. In certain embodiments, the transgene encodes a protein which is not selected from the group consisting of phenylalanine hydroxylase (PAH), iduronate-2-sulfatase (I2S), arylsulfatase A (ARSA), and an anti-complement component 5 antibody.

In certain embodiments, the transgene encodes an antibody or a fragment thereof (e.g., a Fab, scFv, or full-length antibody). Suitable antibodies include, without limitation, muromonab-cd3, efalizumab, tositumomab, daclizumab, nebacumab, catumaxomab, edrecolomab, abciximab, rituximab, basiliximab, palivizumab, infliximab, trastuzumab, adalimumab, ibritumomab tiuxetan, omalizumab, cetuximab, bevacizumab, natalizumab, panitumumab, ranibizumab, eculizumab, certolizumab, ustekinumab, canakinumab, golimumab, ofatumumab, tocilizumab, denosumab, belimumab, ipilimumab, brentuximab vedotin, pertuzumab, raxibacumab, obinutuzumab, alemtuzumab, siltuximab, ramucirumab, vedolizumab, blinatumomab, nivolumab, pembrolizumab, idarucizumab, necitumumab, dinutuximab, secukinumab, mepolizumab, alirocumab, evolocumab, daratumumab, elotuzumab, ixekizumab, reslizumab, olaratumab, bezlotoxumab, atezolizumab, obiltoxaximab, inotuzumab ozogamicin, brodalumab, guselkumab, dupilumab, sarilumab, avelumab, ocrelizumab, emicizumab, benralizumab, gemtuzumab ozogamicin, durvalumab, burosumab, erenumab, galcanezumab, lanadelumab, mogamulizumab, tildrakizumab, cemiplimab, fremanezumab, ravulizumab, emapalumab, ibalizumab, moxetumomab, caplacizumab, romosozumab, risankizumab, polatuzumab, eptinezumab, leronlimab, sacituzumab, brolucizumab, isatuximab, and teprotumumab. In certain embodiments, the transgene encodes an antibody which is not an anti-complement component 5 antibody or a fragment thereof.

In certain embodiments, the transgene encodes a nuclease. Suitable nucleases include, without limitation, zinc fingers nucleases (ZFN) (see, e.g., Porteus and Baltimore (2003) Science 300:763; Miller et al. (2007) Nat. Biotechnol. 25:778-785; Sander et al. (2011) Nature Methods 8:67-69; and Wood et al. (2011) Science 333:307, each of which is hereby incorporated by reference in its entirety), transcription activator-like effectors nucleases (TALEN) (see, e.g., Wood et al. (2011) Science 333:307; Boch et al. (2009) Science 326:1509-1512; Moscou and Bogdanove (2009) Science 326:1501; Christian et al. (2010) Genetics 186:757-761; Miller et al. (2011) Nat. Biotechnol. 29:143-148; Zhang et al. (2011) Nat. Biotechnol. 29:149-153; and Reyon et al. (2012) Nat. Biotechnol. 30 (5): 460-465, each of which is hereby incorporated by reference in its entirety), homing endonucleases, meganucleases (see, e.g., U.S. Patent Publication No. US 2014/0121115, which is hereby incorporated by reference in its entirety), and RNA-guided nucleases (see, e.g., Makarova et al. (2018) The CRISPR Journal 1 (5): 325-336; and Adli (2018) Nat. Communications 9:1911, each of which is hereby incorporated by reference in its entirety).

In certain embodiments, the transgene encodes an RNA-guided nuclease. Suitable RNA-guided nucleases include, without limitation, Class I and Class II clustered regularly interspaced short palindromic repeats (CRISPR)-associated nucleases. Class I is divided into types I, III, and IV, and includes, without limitation, type I (Cas3), type I-A (Cas8a, Cas5), type I-B (Cas8b), type I-C(Cas8c), type 1-D (Cas10d), type I-E (Cse1, Cse2), type I-F (Csy1, Csy2, Csy3), type I-U (GSU0054), type III (Cas10), type III-A (Csm2), type III-B (Cmr5), type III-C(Csx10 or Csx11), type III-D (Csx10), and type IV (Csf1). Class II is divided into types II, V, and VI, and includes, without limitation, type II (Cas9), type II-A (Csn2), type II-B (Cas4), type V (Cpf1, C2c1, C2c3), and type VI (Cas13a, Cas13b, Cas13c). RNA-guided nucleases also include naturally-occurring Class II CRISPR nucleases such as Cas9 (Type II) or Cas12a/Cpf1 (Type V), as well as other nucleases derived or obtained therefrom. Exemplary Cas9 nucleases that may be used in the present invention include, but are not limited to, S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9).

In certain embodiments, the transgene encodes one or more reporter sequences, which produce a detectable signal upon expression. Such reporter sequences include, without limitation, DNA sequences encoding β-lactamase, β-galactosidase (LacZ), alkaline phosphatase, thymidine kinase, green fluorescent protein (GFP), red fluorescent protein (RFP), chloramphenicol acetyltransferase (CAT), luciferase, membrane bound proteins, including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

In certain embodiments, the rAAV2 genome comprises a transcriptional regulatory element (TRE) operably linked to the transgene, to control expression of an RNA or polypeptide encoded by the transgene. In certain embodiments, the TRE comprises a constitutive promoter. In certain embodiments, the TRE can be active in any mammalian cell (e.g., any human cell). In certain embodiments, the TRE is active in a broad range of human cells. Such TREs may comprise constitutive promoter and/or enhancer elements, including any of those described herein, and any of those known to one of skill in the art. In certain embodiments, the TRE comprises an inducible promoter. In certain embodiments, the TRE may be a tissue-specific TRE, i.e., it is active in specific tissue(s) and/or organ(s). A tissue-specific TRE comprises one or more tissue-specific promoter and/or enhancer elements, and optionally one or more constitutive promoter and/or enhancer elements. A skilled artisan would appreciate that tissue-specific promoter and/or enhancer elements can be isolated from genes specifically expressed in the tissue by methods well known in the art.

Suitable promoters include, e.g., cytomegalovirus promoter (CMV) (Stinski et al. (1985) Journal of Virology 55 (2): 431-441), CMV early enhancer/chicken β-actin (CBA) promoter/rabbit β-globin intron (CAG) (Miyazaki et al. (1989) Gene 79 (2): 269-277), CBSB (Jacobson et al. (2006) Molecular Therapy 13 (6): 1074-1084), human elongation factor 1α promoter (EF1α) (Kim et al. (1990) Gene 91 (2): 217-223), human phosphoglycerate kinase promoter (PGK) (Singer-Sam et al. (1984) Gene 32 (3): 409-417), mitochondrial heavy-strand promoter (Lodeiro et al. (2012) PNAS 109 (17): 6513-6518), ubiquitin promoter (Wulff et al. (1990) FEBS Letters 261:101-105).

In certain embodiments, the TRE is brain-specific (e.g., neuron-specific, glial cell-specific, astrocyte-specific, oligodendrocyte-specific, microglia-specific and/or central nervous system-specific). Exemplary brain-specific TREs may comprise one or more elements from, without limitation, human glial fibrillary acidic protein (GFAP) promoter, human synapsin 1 (SYN1) promoter, human synapsin 2 (SYN2) promoter, human metallothionein 3 (MT3) promoter, and/or human proteolipid protein 1 (PLP1) promoter. More brain-specific promoter elements are disclosed in WO 2016/100575A1, which is incorporated by reference herein in its entirety.

In certain embodiments, the native promoter for the transgene may be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In certain embodiments, the rAAV2 particles comprise an editing genome. Editing genomes can be used to edit the genome of a cell by homologous recombination of the editing genome with a genomic region surrounding a target locus in the cell. In certain embodiments, the editing genome is designed to correct a genetic defect in a gene by homologous recombination. Editing genomes generally comprise: (i) an editing element for editing a target locus in a target gene, (ii) a 5′ homology arm nucleotide sequence 5′ of the editing element having homology to a first genomic region 5′ to the target locus, and (iii) a 3′ homology arm nucleotide sequence 3′ of the editing element having homology to a second genomic region 3′ to the target locus, wherein the portion of the editing genome comprising the 5′ homology arm, editing element, and 3′ homology arm can be in the sense or antisense orientation relative to the target locus. Suitable target genes for editing using an editing genome include, without limitation, phenylalanine hydroxylase (PAH), cystic fibrosis conductance transmembrane regulator (CFTR), beta hemoglobin (HBB), oculocutaneous albinism II (OCA2), Huntingtin (HTT), dystrophia myotonica-protein kinase (DMPK), low-density lipoprotein receptor (LDLR), apolipoprotein B (APOB), neurofibromin 1 (NF1), polycystic kidney disease 1 (PKD1), polycystic kidney disease 2 (PKD2), coagulation factor VIII (F8), dystrophin (DMD), phosphate-regulating endopeptidase homologue, X-linked (PHEX), methyl-CpG-binding protein 2 (MECP2), and ubiquitin-specific peptidase 9Y, Y-linked (USP9Y). In certain embodiments, suitable target genes for editing using an editing genome are not selected from the group consisting of phenylalanine hydroxylase, arylsulfatase A, and iduronate 2-sulfatase.

In certain embodiments, the rAAV2 genomes disclosed herein further comprise a transcription terminator (e.g., a polyadenylation sequence). In certain embodiments, the transcription terminator is 3′ to the transgene. The transcription terminator may be any sequence that effectively terminates transcription, and a skilled artisan would appreciate that such sequences can be isolated from any genes that are expressed in the cell in which transcription of the at least a portion of an antibody coding sequence is desired. In certain embodiments, the transcription terminator comprises a polyadenylation sequence. In certain embodiments, the polyadenylation sequence is identical or substantially identical to the endogenous polyadenylation sequence of an immunoglobulin gene. In certain embodiments, the polyadenylation sequence is an exogenous polyadenylation sequence. In certain embodiments, the polyadenylation sequence is an SV40 polyadenylation sequence.

In certain embodiments, the rAAV2 genomes disclosed herein further comprise a 5′ inverted terminal repeat (5′ ITR) nucleotide sequence 5′ of the TRE, and a 3′ inverted terminal repeat (3′ ITR) nucleotide sequence 3′ of the polyadenylation sequence associated with an antibody light chain coding sequence. ITR sequences from any AAV2 serotype or variant thereof can be used in the rAAV2 genomes disclosed herein. The 5′ and 3′ ITR can be from an AAV2 of the same serotype or from AAV2s of different serotypes.

In certain embodiments, the 5′ ITR or 3′ ITR is from AAV2. In certain embodiments, both the 5′ ITR and the 3′ ITR are from AAV2. In certain embodiments, the 5′ ITR nucleotide sequence and the 3′ ITR nucleotide sequence are substantially complementary to each other (e.g., are complementary to each other except for mismatch at 1, 2, 3, 4, or 5 nucleotide positions in the 5′ or 3′ ITR).

In certain embodiments, the 5′ ITR or the 3′ ITR is modified to reduce or abolish resolution by Rep protein (“non-resolvable ITR”). In certain embodiments, the non-resolvable ITR comprises an insertion, deletion, or substitution in the nucleotide sequence of the terminal resolution site. Such modification allows formation of a self-complementary, double-stranded DNA genome of the AAV2 after the rAAV2 genome is replicated in an infected cell. Exemplary non-resolvable ITR sequences are known in the art (see, e.g., those provided in U.S. Pat. Nos. 7,790,154 and 9,783,824, which are incorporated by reference herein in their entirety).

In certain embodiments, the 5′ ITR is flanked by an additional nucleotide sequence derived from a wild-type AAV2 genomic sequence. In certain embodiments, the 5′ ITR is flanked by an additional 46 bp sequence derived from a wild-type AAV2 sequence that is adjacent to a wild-type AAV2 ITR in an AAV2 genome. In certain embodiments, the additional 46 bp sequence is 3′ to the 5′ ITR in the rAAV genome.

In certain embodiments, the 3′ ITR is flanked by an additional nucleotide sequence derived from a wild-type AAV2 genomic sequence. In certain embodiments, the 3′ ITR is flanked by an additional 37 bp sequence derived from a wild-type AAV2 sequence that is adjacent to a wild-type AAV2 ITR in an AAV2 genome. See, e.g., Savy et al., Human Gene Therapy Methods (2017) 28 (5): 277-289 (which is hereby incorporated by reference herein in its entirety). In certain embodiments, the additional 37 bp sequence is 5′ to the 3′ ITR in the rAAV2 genome.

### AAV2 Capsid Protein and Cap Gene Products

In certain embodiments, the AAV2 particles in the composition comprise AAV2 capsid proteins. The AAV2 capsid protein can be selected from any AAV2 capsid known in the art, including capsid proteins from natural AAV2 isolates and variants thereof. AAV2 capsid proteins, encoded by the Cap gene, include VP1, VP2, and VP3 capsid proteins. VP1, VP2, and/or VP3 capsid proteins assemble into a particle that surrounds the rAAV2 genome. In certain embodiments, assembly of the capsid proteins is facilitated by the assembly-activating protein (AAP). Particles of AAV2 require the role of AAP in transporting the capsid proteins to the nucleolus for assembly. The sequences of the various AAV2 capsid proteins are disclosed in, e.g., U.S. Patent Publication Nos.: US20140359799, US20150376607, US20150159173, US20170081680, and US20170360962A1, and PCT Publication No. WO2020227515, the disclosures of which are incorporated by reference herein in their entireties.

### AAV2 Rep Genes and Rep Gene Products

In certain embodiments, the AAV2 particles in the composition comprise AAV2 replication proteins. As used throughout, a Rep gene is a nucleic acid sequence encoding one or more replication (Rep) proteins. The gene can comprise a coding and/or a non-coding sequence from the Rep gene of an AAV2 genome. As used throughout, a gene can include exonic regions, intronic regions, and/or untranslated regions from a genomic sequence. In some cases, a Rep gene may have one or more coding sequences due to alternative splicing or alternative translation initiation, etc. A coding sequence may be a wild-type or a non-naturally occurring coding sequence (e.g., a codon optimized sequence encoding Rep).

The Rep gene encodes Rep proteins (e.g., Rep78, Rep68, Rep52 and Rep40_that are involved in AAV genome replication and packaging of the viral genome. During native AAV2 replication, expression of Rep proteins is controlled by the p5 and p19 promoters. The p5 promoter drives expression of the alternative splice variants Rep78 and Rep68. The p19 promoter drives expression of the alternative splice variants Rep52 and Rep40. A Rep nucleic acid encoding one or more Rep proteins can be derived from AAV2. An exemplary AAV2 genome sequence is available via NCBI Reference Sequence NC_001401.2. According to the NCBI Reference Sequence, Rep68 is encoded by nucleotides 321 to 2252; Rep78 is encoded by nucleotides 321 to 2186; Rep40 is encoded by nucleotides 993 to 2252; and Rep52 is encoded by nucleotides 993 to 2186. In certain embodiments, the present disclosure provides a nucleic acid comprising a nucleotide sequence corresponding to the sequence encoding Rep40, Rep68, Rep78, Rep52 as described for AAV2, in a different adenovirus serotype, for example, AAV5.

### AAV2 Vectors and Vector Systems

Vector systems (i.e., at least two vectors) are used to produce the AAV2 viral particles in the compositions described herein. The vector system comprises one or more Rep genes (e.g., AAV2 Rep) and one or more Cap genes (e.g., AAV2 Cap). The polynucleotides comprising these genes may be juxtaposed on the same vector and operably linked to the same promoter. Optionally these genes can be split by an intervening sequence, by the use of a different start codon or a different promoter, and/or by placement on different vectors.

A helper vector is also required for packaging recombinant AAV2 particles. Typically, adenoviral (AdV) helper factors E1A, E1B, E2A, E4, and VA RNA are necessary for viral replication; however, producer cells may express ELA gene product and E1B such that these need not be encoded by the helper vector. Any of the helper virus factors described herein can be from an adenoviral (AdV) genome, for example, from an AdV type 2 genome or from an AdV type 5 genome. Nucleic acids encoding these AdV helper factors or a portion thereof (i.e., E1A, E1B, E2A, E4, and/or VA RNA) can be transfected into a host cell on a single vector. Alternatively, one or more vectors comprising these helper factors can be transfected into a host cell. The helper factors can also be expressed by transfecting, into a host cell, one or more vectors encoding helper factors that are not endogenously expressed by the host cell. In some instances, certain host cells such as, for example, HEK293T cells, endogenously provide some, but not all, required helper factors, and the remaining helper factors can be provided exogenously via vector transfection. For example, HEK293T cells endogenously express AdV E1A and E1B genes and can be transfected with a vector comprising one or more AdV E4 gene products or fragments thereof, one or more E2a gene products or fragments thereof, and virus-associated (VA) RNA to produce the required helper elements for AAV2 production, e.g., E1A, E1B, E2A, one or more E4 gene products, and VA RNA in the host cell.

In any of the helper vectors described herein, a nucleic acid sequence encoding one or more of the helper factors or a functional portion thereof for AAV2 replication, e.g., an ELA gene product, an E1B gene product, an E4 gene product, an E2a gene product, and/or VA RNA, can be operably linked to a transcriptional regulatory element that controls the expression of the helper factor. In certain embodiments, the transcriptional regulatory element comprises a promoter (e.g., a constitutive promoter, an inducible promoter, or a native promoter). Suitable promoters are known to those of skill in the art and include, without limitation, an RSV LTR promoter, a CMV immediate early promoter, an SV40 promoter, a dihydrofolate reductase promoter, a cytoplasmic β-actin promoter, a phosphoglycerate kinase (PGK) promoter, a metallothionine (MT) promoter, a mouse mammary tumor virus (MMTV) promoter, a T7 promoter, an ecdysone insect promoter, a tetracycline-repressible promoter, a tetracycline-inducible promoter, an RU486-inducible promoter, and a rapamycin-inducible promoter.

AAV2 vector systems as described herein can comprise 2, 3, 4, or more plasmids comprising polynucleotide sequences (i.e., genes) encoding the proteins as described herein. By way of example, a first vector may comprise the Rep and Cap genes, a second vector may comprise the transgene, and a third helper vector may comprise packaging genes; a first vector may comprise the Rep, a second vector may comprise the Cap genes, a third vector may comprise the transgene, and a fourth helper vector may comprise packaging genes; or a first vector may comprise the Rep and Cap genes and the transgene and a second vector may be the helper vector.

Optionally, the AAV2 vector system used to produce the AAV2 particles of the composition comprises at least two vectors and split Rep and Cap genes, wherein a first vector comprises a gene of interest and a Cap gene and wherein the Rep and Cap genes have different start codons and/or different promoters. In some systems, the first vector further comprises the Rep gene, optionally with an intervening nucleotide sequence between the Rep and Cap genes, and in some systems the Rep and Cap genes are on different vectors. As used herein, intervening sequences placed between Rep and Cap genes refers to a portion of the plasmid backbone between separate expression cassette for Rep and Cap.

As used throughout, a vector (also referred to herein as a plasmid) refers to a nucleic acid construct (i.e., a polynucleotide). A nucleic acid construct that comprises one or more polynucleotides that encode one or more elements required for AAV2 production can be used as a vector or as a component of the vector systems described herein. Suitable vectors, include, without limitation, plasmids, minimal vectors (e.g., minicircles, Nanoplasmids™, doggybones, MIDGE vectors, and the like), viruses, cosmids, artificial chromosomes, linear DNA, and mRNA. Suitable DNA minimal vectors include, without limitation, linear covalently closed DNA (e.g., ministring DNA), linear covalently closed dumbbell shaped DNA (e.g., doggybone DNA, dumbbell DNA), minicircles, Nanoplasmids™, minimalistic immunologically defined gene expression (MIDGE) vectors, and others known to those of skill in the art. DNA minimal vectors and their methods of production are described in, e.g., U.S. Patent Application Publication Nos. 20100233814, 20120282283, 20130216562, 20150218565, 20150218586, 20160008488, 20160215296, 20160355827, 20190185924, 20200277624, and 20210010021, all of which are herein incorporated by reference in their entireties.

In some embodiments, a vector is a circular, single stranded or double stranded nucleic acid sequence construct, e.g., a double-stranded DNA plasmid. In some cases, a vector is an extrachromosomal circular DNA comprising one or more origins of replication capable of autonomous replication in a given cell, for example, a eukaryotic cell or a bacterial cell. In some embodiments, a vector does not comprise one or more sequences necessary for autonomous replication in bacterial cells, for example, a bacterial origin of replication. Optionally, a vector comprises one or more elements required for AAV2 replication. In some embodiments, a vector system comprises a vector or combination of vectors comprising the elements required for AAV2 replication. In some embodiments, a vector system comprises a vector comprising the elements required for AAV2 replication and a vector comprising a transgene or gene or interest (GOI).

As used herein, the term nucleic acid or nucleotide sequence or polynucleotide refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar properties as the reference nucleic acid. A nucleic acid sequence can comprise combinations of deoxyribonucleic acids and ribonucleic acids. Such deoxyribonucleic acids and ribonucleic acids include both naturally occurring molecules and synthetic analogues. Nucleic acids also encompass all forms of sequences including, but not limited to, single-stranded forms, double-stranded forms, hairpins, stem-and-loop structures, and the like.

In some embodiments, the nucleic acids in the vectors described herein are optimized, e.g., by codon/RNA optimization, replacement with heterologous signal sequences, with enhancers for the promoter sequences, with non-native start codons, and/or elimination of mRNA instability elements. Methods to generate optimized polynucleotides for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly, all of which are herein incorporated by reference in their entireties. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In certain embodiments, it can be desirable to alter one or more codons to encode a conservative mutation, e.g., a similar amino acid with similar chemical structure and properties and/or function as the original amino acid.

### Buffer

In one embodiment, the buffers of the AAV2 compositions can comprise an effective amount of an additive for reducing aggregation, a buffering agent, a salt, a surfactant, and optionally other excipients.

### Additives

Additives in the buffers of the compositions described herein are selected for their ability to reduce AAV2 aggregation. However, the concentration of the additives is generally higher than used in commercial manufacturing of AAV compositions. The additives can be excluded or included additives, which work through different mechanisms to reduce aggregation. Excluded additives are understood to prevent aggregation by preventing motion of molecules. Such excluded additives useful in the compositions and methods described herein include trehalose, sucrose, or glycerol. Included additives are understood to prevent aggregation by interacting with the surface of AAV2 particles to solubilize potential aggregation sites on the particle surface. Examples of included additives are monovalent sodium salts, urea, guanidine. Trehalose is used throughout as an exemplary additive.

Critical to the composition is that the additive (e.g., trehalose) be present in the buffer and composition at a concentration high enough to sufficiently reduce aggregation and at a concentration low enough to avoid unacceptable consequences. Thus, the composition optionally comprises an aggregation reduction agent (e.g., trehalose) at a concentration of greater than 5% (w/v). Optionally, the concentration is greater than 6% (w/v), greater than 7% (w/v), or greater than 8% (w/v). For example, the concentration of the agent is optionally 6-11% (w/v) or 7-8% (w/v/).

### Buffering Agents

In certain embodiments, the composition comprises a buffering agent. Acceptable buffering agents are well known in the art, including, without intending to be limiting: histidine (e.g., L-histidine), histidine hydrochloride (histidine-HCl), sodium phosphate dibasic, sodium chloride, sodium phosphate dibasic anhydrous, sodium phosphate hexahydrate, sodium phosphate monobasic monohydrate, sodium phosphate, sodium succinate, sodium acetate, sodium carbonate, sodium sulfate, magnesium sulfate, magnesium chloride, tromethamine, tris(hydroxymethyl)aminomethane hydrochloride (Tris-HCl), bicine, glycine, glycyl glycine, lysine, arginine, 4-(2-hydroxyethyl)-1-piperazineethanesulfonic acid (HEPES), calcium sulfate, calcium chloride, calcium citrate, and any mixtures thereof. Optionally, the buffering agent is neither a citrate nor a potassium salt or other potassium species.

In certain embodiments, the buffering agent is histidine, which is the exemplary buffering agent used in the Examples. The composition comprises about 1 mM to about 25 mM histidine, for example, about 10 mM to about 20 mM, about 15 mM to about 20 mM, and about 17-18 mM histidine.

### Salts

The buffers and compositions described herein can further comprise a salt, e.g., sodium chloride, magnesium chloride, calcium chloride, and any mixtures thereof. Optionally, the salt is not potassium chloride. Sodium chloride is the exemplary salt used in the Examples, but sodium chloride can be substituted for another salt, or a mixture of suitable salts, adjusted to the appropriate concentration to provide an ionic strength equivalent to that provided by the sodium chloride. In certain embodiments, the composition comprises about 50 mM to about 200 mM of the salt, for example, 150 mM to about 190 mM, about 170 mM to about 200 mM. By way of example, the composition can comprise about 175 mM sodium chloride. Salt concentrations referred to herein may also refer to an equivalent ionic strength provided by the salt concentration and may be referred to as or an ionic strength equivalent thereto.

### Surfactants

In certain embodiments, the composition comprises a surfactant (e.g., a non-ionic surfactant). Acceptable surfactants are known in the art, including, without limitation, Poloxamer 188, Polysorbate 20, Polysorbate 80, Brij-35, and any mixtures thereof. Poloxamer 188 (P188), an exemplary surfactant used in the Examples below, can be replaced with a different surfactant.

Optionally, the composition comprises about 0.0001% (w/v) to about 1% (w/v) Poloxamer 188, for example, about 0.001-0.03% (w/v) Poloxamer 188. Optionally, the final product comprises 0.03% (w/v) Poloxamer 188, whereas the chromatography product comprises 0.001% (w/v) Poloxamer 188.

### Methods of Use

In certain embodiments, the present disclosure provides a method for transducing a cell (in vivo or in vitro) and methods of treating a subject with the product of a transgene. The methods generally comprise administering to the cell or the subject an effective amount of an AAV2 composition described herein.

The compositions as described herein can be administered to a subject (e.g., a human subject) by any appropriate route, including, without limitation, intravenous, intraperitoneal, subcutaneous, intramuscular, intrathecal, intranasal, intracerebroventricular, topical or intradermal routes. In certain embodiments, the compositions provided by the present disclosure are suitable for administration via intravenous injection or subcutaneous injection.

The compositions provided by the present disclosure comprise rAAV2 particles. The rAAV2 particles can comprise a transgene, optionally under the control of a transcriptional regulatory element (TRE). Accordingly, in certain embodiments, the present disclosure provides methods for expressing a transgene in a cell (e.g., in the cell of a recipient subject). The method generally comprises administering to the subject an effective amount of a composition comprising an rAAV2 composition as described herein, whereby the cell is transduced by the rAAV2 and the transgene is expressed. The transgene can encode a polypeptide and/or an RNA molecule, as described herein. Accordingly, in certain embodiments, the present disclosure provides methods for producing a polypeptide and/or an RNA molecule in a cell (in vivo or in vitro) by administering to the cell or to the subject an effective amount of a composition comprising an rAAV2 as described herein, whereby the cell is transduced by the rAAV2 and the polypeptide and/or an RNA molecule is produced.

In certain embodiments, the method further comprises the step of storing the composition at a temperature from about −80° C. to about 40° C. For example, the temperature can be from about 2° C. to about 8° C. In some embodiments, the temperature is about 5° C. In some embodiments, the temperature is about 25° C. In some embodiments, the temperature is about 40° C.

### Additional Definitions

As used herein, the term recombinant adeno-associated virus or rAAV refers to an adeno-associated virus (AAV) that is not a wild-type AAV.

As used herein, the term rAAV genome refers to one or more nucleic acid molecules (i.e. polynucleotides) comprising the genome sequence of an rAAV. The skilled artisan will appreciate that where an rAAV genome comprises a transgene, the rAAV genome can be in the sense or antisense orientation relative to the direction of transcription of the transgene.

The use of any and all examples or exemplary language (e.g., such as) provided herein, is intended merely to better illustrate the invention and does not pose a limitation on the scope of the invention unless otherwise claimed.

The terms may, may be, can, and can be, and related terms are intended to convey that the subject matter involved is optional (that is, the subject matter is present in some examples and is not present in other examples), not a reference to a capability of the subject matter or to a probability, unless the context clearly indicates otherwise.

The term about, is used to provide flexibility to a numerical range endpoint by providing that a given value may be slightly above or slightly below the endpoint without affecting the desired result. For example, about can refer to a value that is within +1%, 2%, 3%, 4%, or 5% of the target or reference value.

The terms optional and optionally mean that the subsequently described event, circumstance, or material may or may not occur or be present, and that the description includes instances where the event, circumstance, or material occurs or is present as well as instances where it does not occur or is not present.

The use herein of the terms including, comprising, or having, and variations thereof, is meant to encompass the elements listed thereafter and equivalents thereof as well as additional elements. Embodiments recited as including, comprising, or having certain elements are also contemplated as consisting essentially of and consisting of those certain elements. As used herein, and/or refers to and encompasses any and all possible combinations of one or more of the associated listed items, as well as the lack of combinations where interpreted in the alternative (“or”).

### EXAMPLES

The following Examples are offered by way of illustration and not by way of limitation.

### Example 1: Optimization of AAV2 Formulations by Front-End Dilution/Concentration

The present example relates to processes that reduce AAV2 aggregation in an AAV2 composition by optimizing buffers utilized in the concentration and purification stage following AAV2 recovery. Particularly, an AEX chromatography product was front-end diluted prior to the UFDF process and the level of aggregation in the UFDF product was measured using an UNCLE instrument from Unchained Labs that utilizes dynamic light scattering (DLS) in real time. DLS measures light scattering intensity over time and can correlate it to a diffusion coefficient and hydrodynamic diameter (nm). DLS can also detect even very rare aggregates in a sample, since large particles scatter light intensity.

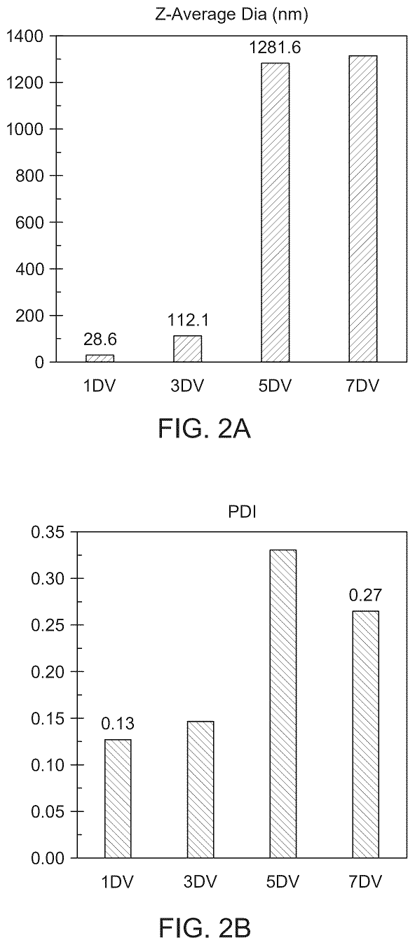

Table 1 shows AAV serotype aggregation in a Formulation C buffer (i.e., a control buffer containing on 3% (w/v) trehalose). Most AAV2 serotypes are stable in the control buffer which comprises 20 nM histidine, 175 mM sodium chloride (NaCl), 3% (w/v %) trehalose, and 0.001% Poloxamer-188. But, AAV2 aggregation was observed using this buffer. AAV2 after UFDF showed high aggregation during diafiltration; at 3 diavolumes (3DV) particle size was >100 nm. FIG. 2A-2B are plots showing aggregation of AAV2 particles during diafiltration. FIG. 2A is a plot of average diameter with increasing diavolumes whereas FIG. 2B is a plot of polydispersity index (PDI) with increasing diavolumes.

TABLE 1
Serotype Aggregation in a Control Buffer

| Serotype | Z-Average Diameter (nm) | Polydispersity Index (PDI) |
|---|---|---|
| AAV1 | 31.41 | 0.231 |
| AAV2 | 407.55 | 0.369 |
| AAV5 | 27.71 | 0.139 |
| AAV6 | 30.59 | 0.211 |
| AAV8 | 32.75 | 0.224 |
| AAV9 | 26.79 | 0.052 |
| AAVrh10 | 29.42 | 0.134 |
| AAVrh74 | 29.94 | 0.143 |

To reduce the level of aggregation, the process was modified as shown in FIG. 3. The chromatography product was diluted with a pre-formulation buffer having a higher level of trehalose. The pre-formulation buffer comprised of 20 mM histidine, 175 mM NaCl, 8% (w/v %) trehalose, and 0.001% (w/v %) Poloxamer-188, with a pH of 7.3. Following introduction of the modified buffer to the chromatography product, the final concentration of histidine and trehalose in the diluted chromatography product was 17 mM histidine and 7% trehalose. The sheer rate of the process of FIG. 3 was 4000 sec-1 and TMP 6-8 PSI. A 1.7× chase strategy was employed to avoid air-liquid interface. That is, buffer was used to recover the system hold up volume. Further, sheer rate was dropped to 500 sec-1 during the final recovery step. A final target concentration of 1×1013 vg/mL was chosen.

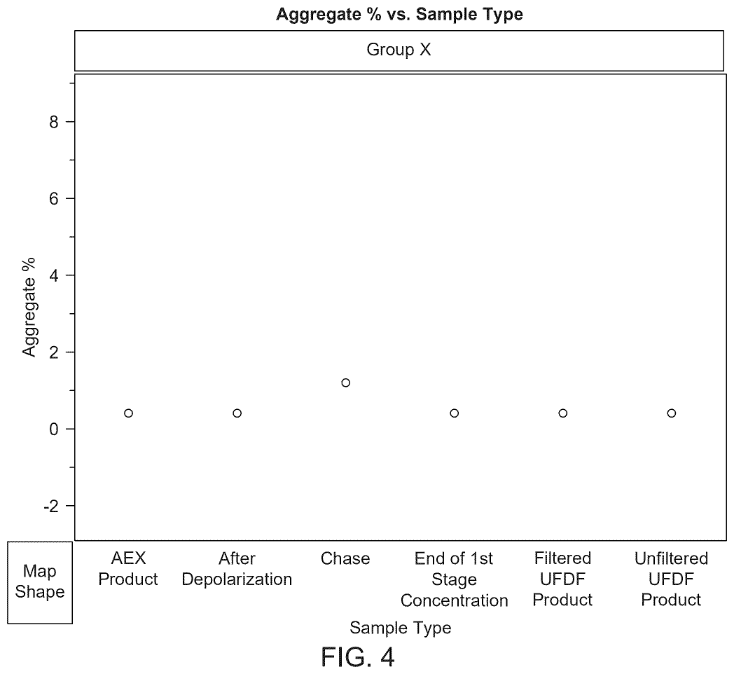

Analysis of DLS data after the process of FIG. 3 showed no measurable aggregation. Size-exclusion chromatography (SEC) data also showed no substantial aggregation with the modified process. FIG. 4 shows the level of aggregation by sample type at each step of the process according to FIG. 3. Comparable percentages of aggregates were observed across the entire process. As further illustrated in Table 2, no SEC aggregation was seen throughout the process and high VG and particle yield was observed for the process.

Samples were taken and analyzed for VG and Particle mass recovery via ddPCR and Particle ELISA, respectively. A summary of the titers achieved and the subsequent recoveries can be found in Table 2. The UFDF Product achieved a final titer of 9.82×1012 VG/mL and 2.81×1013 particles/mL prior to sterile filtration. VG and Particle yields were 99% and 91%, respectively. This data demonstrates that the methodology and formulation used in this study is capable of delivering a best-in-class VG titer and recovery through UFDF for AAV2 particles. This shows high % viral genome (VG) and particle yield of the UFDF product (before and after filtration) can be achieved using the method of FIG. 3.

TABLE 2
Vector Genome (VG) and Particle Yield Across the Purification Stream

| Sample | | VG/mL | | % VG | Particles/mL | Total | % Particle |
|---|---|---|---|---|---|---|---|
| Name | Vol. | (ddPCR) | Total VGs | Recovery | (ELISA) | Particles | Recovery |
| Diluted | 64 | 3.29E+12 | 2.11E+14 | N/A | 1.02E+13 | 6.53E+14 | N/A |
| AEX | | | | | | | |
| Product | | | | | | | |
| (before | | | | | | | |
| UFDF) | | | | | | | |
| UFDF | 21.2 | 9.82E+12 | 2.08E+14 | 99% | 2.81E+13 | 5.96E+14 | 91% |
| Product | | | | | | | |
| Before | | | | | | | |
| Sterile | | | | | | | |
| Filtration | | | | | | | |
| Final | 21.9 | 9.52E+12 | 2.08E+14 | 99% | 2.83E+13 | 6.20E+14 | 95% |
| UFDF | | | | | | | |
| Product | | | | | | | |
| (Post | | | | | | | |
| Sterile | | | | | | | |
| Filtration) | | | | | | | |

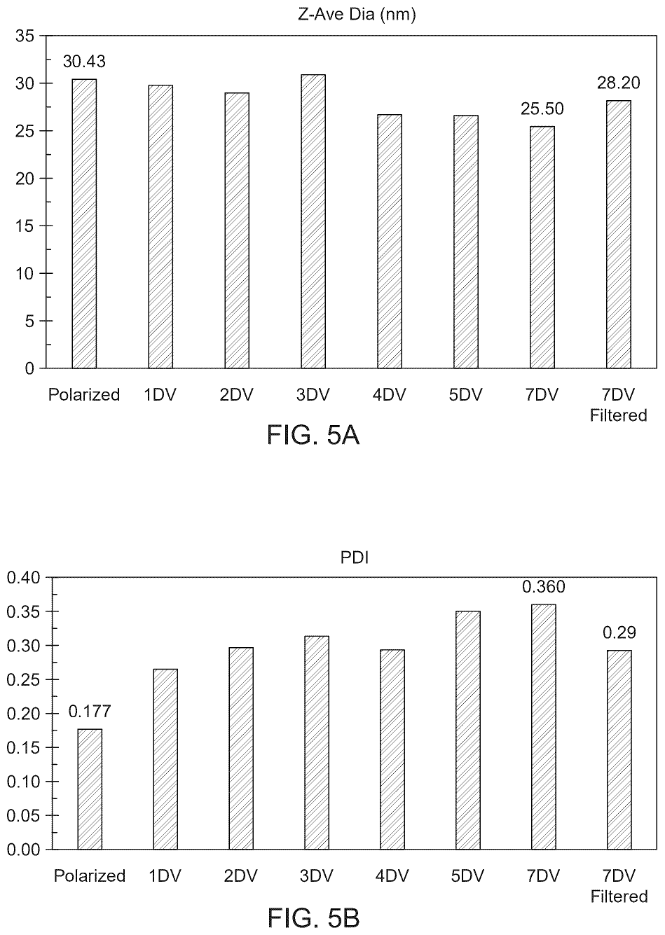

Additionally, as shown in FIG. 5A and FIG. 5B, using DLS, aggregation levels as measured with DLS were low in the presence of high trehalose throughout the diafiltration process.

Substantial aggregation was observed, however, with dilution and diafiltration in a high salt buffer (20 mM histidine, 300 mM NaCl, 3% (w/v %) trehalose, 0.001% (w/v %) Poloxamer 188, pH 7.3) and a Tris buffer containing glycine (47.5 mM Tris, 95-100 mM glycine, and 100 mM sodium chloride. Similarly, use of an AEX neutralization buffer (100 mM glycine, 100 mM NaCl, pH 2.7; neutralization: 5% (v/v %) 1M Tris, 100 mM NaCl, pH 10.9; buffer: 47.5 mM Tris, 95 mM glycine, 100 mM NaCl, pH 8) also resulted in marked aggregation by 7DV.

### Example 2: AAV2 Stability with High Trehalose Across the UFDF Process

An 8 mL sample of predominantly full (DNA-containing) AAV2 particles generated through an AEX step was diluted 1:7 with pre-formulation buffer (1 part sample, 7 parts buffer) where the pre-formulation buffer used was 20 mM Histidine, 175 mM Sodium Chloride, 8% (w/v %) Trehalose, 0.001% (w/v %) Poloxamer 188 pH 7.3. The diluted sample (64 milliliters) was fed into and simultaneously concentrated in a retentate vessel (50 mL conical tube) connected to a 20 cm2 Microkros 100 kDa MPES hollow fiber membrane utilizing a sheer rate of 4,000 s-1. The TMP was maintained during concentration between 6 and 8 psi. The concentration was performed until the sample volume was 8 milliliters. Once the sample was concentrated to 8 milliliters, the same pre-formulation buffer was fed into the retentate vessel at a volumetric flow-rate to maintain the volume in the UFDF system to 8 milliliters. This was performed until the volume of sample in the retentate vessel was buffer exchanged seven times. At the end of the buffer exchange (diafiltration), the sample in the retentate vessel was removed without removing the sample held up in the rest of the system. A volume of pre-formulation buffer equivalent to 1.7× the system hold-up volume was pipetted into the retentate vessel and the pump was turned on to circulate and depolarize the material in the membrane at a constant sheer rate of 500s-1 for 5 minutes. This is known as the “chase.” At the end of the five minutes, the chase sample in the retentate and system hold-up was removed and pooled with the previously collected retentate. This was known as the UFDF product.

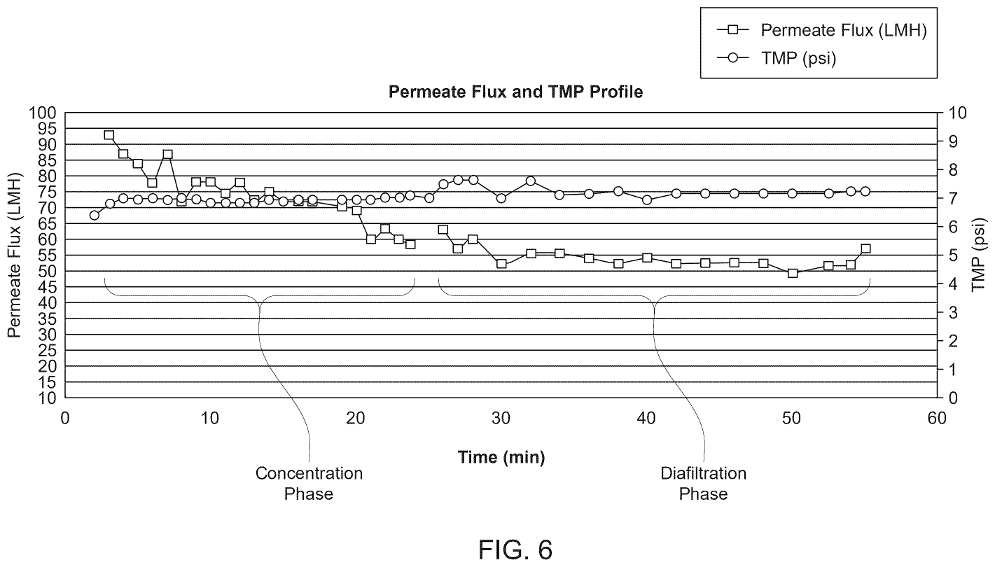

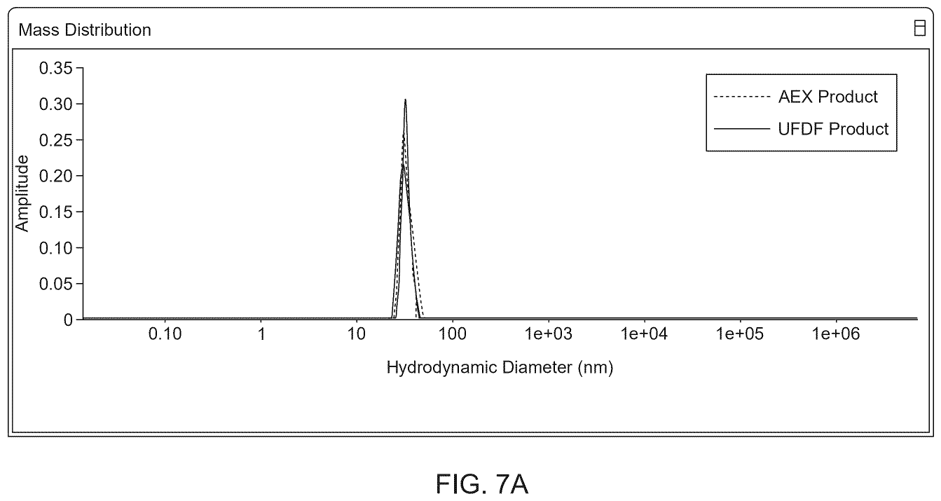

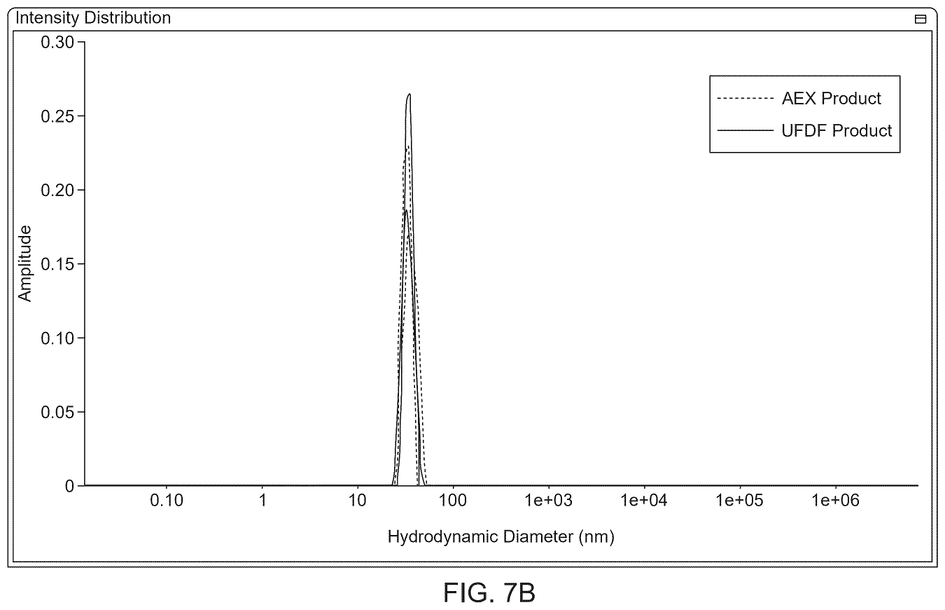

The permeate flux was recorded and analyzed during the concentration and buffer exchange steps. See FIG. 6. A 30% flux decay was observed during the concentration phase. No significant flux decay was observed during the diafiltration phase. As shown in FIGS. 7A and 7B, the average peak diameter (nm) and PDI were found to be comparable between AEX and UFDF products as measured with DLS.

### Example 3: AAV2 Stability Over Time

UFDF AAV2 products were prepared as described in Example 2, using pre-formulation buffer comprising 20 mM Histidine, 175 mM Sodium Chloride, 0.001% (w/v %) Poloxamer 188 pH 7.3, and 3%, 5%, or 8% (w/v %) trehalose, 5% (w/v %) sucrose, or 5% (w/v %) glycerol. DLS was used to evaluate aggregation at 25° C. or 40° C. immediately after diafiltration and at 6 and 8 days post-diafiltration. Results are shown in Tables 3 and 4.

TABLE 3
Hydrodynamic Diameter Time Course

| Hydrodynamic | Time (Days) at 25° C. | | | Time (Days) at 40° C. | | |
|---|---|---|---|---|---|---|
| Diameter (nm) | 0 | 6 | 8 | 0 | 6 | 8 |
| 3% Trehalose | 28.90 | 29.96 | 29.99 | 28.90 | 31.96 | 32.38 |
| 5% Trehalose | 28.86 | 30.23 | 29.51 | 28.86 | 31.13 | 31.90 |
| 8% Trehalose | 29.41 | 30.19 | 30.72 | 29.41 | 33.68 | 33.66 |
| 5% Sucrose | 29.37 | 30.94 | 29.88 | 29.37 | 31.73 | 31.90 |
| 5% Glycerol | 29.81 | 31.29 | 30.97 | 29.81 | 33.13 | 34.21 |

TABLE 4
Polydispersity Index Time Course

| Polydispersity | Time (Days) at 25° C. | | | Time (Days) at 40° C. | | |
|---|---|---|---|---|---|---|
| Index (PDI) | 0 | 6 | 8 | 0 | 6 | 8 |
| 3% Trehalose | 0.01 | 0.03 | 0.10 | 0.01 | 0.07 | 0.08 |
| 5% Trehalose | 0.01 | 0.01 | 0.04 | 0.01 | 0.08 | 0.05 |
| 8% Trehalose | 0.01 | 0.02 | 0.01 | 0.01 | 0.16 | 0.02 |
| 5% Sucrose | 0.01 | 0.01 | 0.03 | 0.01 | 0.12 | 0.11 |
| 5% Glycerol | 0.05 | 0.02 | 0.03 | 0.05 | 0.07 | 0.09 |

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes.

### ADDITIONAL EMBODIMENTS

1. An adenovirus-associated virus type 2 (AAV2) composition comprising:
(a) AAV2 particles containing a transgene; and
(b) a buffer comprising an aggregation-reducing additive of greater than 5% w/v;
wherein the additive is trehalose and wherein the AAV2 composition has less particle aggregation than a control AAV2 composition in a buffer lacking the additive.
   2. The AAV2 composition of embodiment 1, wherein the concentration of the additive in the buffer is greater than 6%, greater than 7%, or 7-8%.
   3. The AAV2 composition of embodiment 1 or 2, wherein the concentration of the additive in the buffer is less than 25%, less than 15%, or less than 10%.
   4. The AAV2 composition of any one of embodiment 1-3, wherein the buffer further comprises histidine.
   5. The AAV2 composition of embodiment 4, wherein concentration of histidine in the buffer is 15-25 mM.
   6. The AAV2 composition of embodiment 5, wherein concentration of histidine in the buffer is 17-18 mM.
   7. The AAV2 composition of any one of embodiments 1-6, wherein the buffer further comprises a salt.
   8. The AAV2 composition of embodiment 7, wherein the salt is sodium chloride.
   9. The AAV2 composition of embodiment 8, wherein concentration of the sodium chloride is 160-190 mM.
   10. The AAV2 composition of any one of embodiments 1-9, wherein the buffer further comprises a surfactant.
   11. The AAV2 composition of embodiment 10, wherein the surfactant is Poloxamer-188.
   12. The AAV2 composition of embodiment 11, wherein the buffer comprises Poloxamer-188 at a concentration of 0.0005-0.0015 (w/v %).
   13. The AAV2 composition of embodiments 1-12, wherein the AAV2 composition has a pH of 7.3.
   14. The AAV2 composition of any one of embodiments 1-13, wherein the AAV2 composition is a chromatography product.
   15. The AAV2 composition of any one of embodiments 1-13, wherein the AAV2 composition is an ultrafiltration/diafiltration product.
   16. The AAV2 composition of any one of embodiments 1-15, wherein aggregation is measured by dynamic light scattering.
   17. The AAV2 composition of embodiment 16, wherein the composition is an ultrafiltration product and wherein aggregation is detected at less than 100 nM or less than 50 nM Z-average diameter with dynamic light scattering after 3 diavolumes for diafiltration.
   18. The AAV2 composition of embodiment 16 or 17, wherein the PDI is less than 0.3.
   19. The AAV2 composition of any one of embodiments 1-15, wherein aggregation is detected by size exclusion chromatography.
   20. The AAV2 composition of embodiment 19, wherein a percentage of aggregated particles detected with size exclusion chromatography is less than 1.0% or less than 0.5%.
   21. The AAV2 composition of any one of embodiments 1-26, wherein the AAV2 composition comprises greater than 1×10¹³ vg/ml.
   22. The AAV2 composition of embodiment 21, wherein the AAV2 composition comprises 5-8×10¹³ vg/ml.
   23. The AAV2 composition of embodiment 22, wherein the AAV2 composition comprises 7-8×10¹³ vg/ml.
   24. The AAV2 composition of any one of embodiments 1-23, wherein the composition is stable during filtration/diafiltration up to 2, 3, 4, 5, 6, or 7 diavolumes.
   25. The AAV2 composition of any one of claims 1-30, wherein the composition is stable for at least one month, one year, or two years at 2-8° C.
   26. An adenovirus-associated virus type 2 (AAV2) composition comprising:
(a) AAV2 particles containing a transgene; and
(b) a buffer comprising an aggregation-reducing additive of greater than 5% w/v;
wherein the additive is sucrose or glycerol and wherein the AAV2 composition has less particle aggregation than a control AAV2 composition in a buffer lacking the additive.
   27. The AAV2 composition of embodiment 26, wherein the concentration of the additive in the buffer is greater than 6%, greater than 7%, or 7-8%.
   28. The AAV2 composition of embodiment 26 or 27, wherein the concentration of the additive in the buffer is less than 25%, less than 15%, or less than 10%.
   29. The AAV2 composition of any one of embodiment 26-28, wherein the buffer further comprises histidine.
   30. The AAV2 composition of embodiment 29, wherein concentration of histidine in the buffer is 15-25 mM.
   31. The AAV2 composition of embodiment 30, wherein concentration of histidine in the buffer is 17-18 mM.
   32. The AAV2 composition of any one of embodiments 26-31, wherein the buffer further comprises a salt.
   33. The AAV2 composition of embodiment 32, wherein the salt is sodium chloride.
   34. The AAV2 composition of embodiment 33, wherein concentration of the sodium chloride is 160-190 mM.
   35. The AAV2 composition of any one of embodiments 26-34, wherein the buffer further comprises a surfactant.
   36. The AAV2 composition of embodiment 35, wherein the surfactant is Poloxamer-188.
   37. The AAV2 composition of embodiment 36, wherein the buffer comprises Poloxamer-188 at a concentration of 0.0005-0.0015 (w/v %).
   38. The AAV2 composition of embodiments 26-37, wherein the AAV2 composition has a pH of 7.3.
   39. The AAV2 composition of any one of embodiments 26-38, wherein the AAV2 composition is a chromatography product.
   40. The AAV2 composition of any one of embodiments 26-38, wherein the AAV2 composition is an ultrafiltration/diafiltration product.
   41. The AAV2 composition of any one of embodiments 26-40, wherein aggregation is measured by dynamic light scattering.
   42. The AAV2 composition of embodiment 41, wherein aggregation is detected at less than 100 nM or less than 50 nM Z-average diameter with dynamic light scattering after 3 diavolumes for diafiltration.
   43. The AAV2 composition of embodiment 41 or 42, wherein the PDI is less than 0.3.
   44. The AAV2 composition of any one of embodiments 26-40, wherein aggregation is detected by size exclusion chromatography.
   45. The AAV2 composition of embodiment 44, wherein a percentage of aggregated particles detected with size exclusion chromatography is less than 1.0% or less than 0.5%.
   46. The AAV2 composition of any one of embodiments 2-45, wherein the AAV2 composition comprises greater than 1×10¹³ vg/ml.
   47. The AAV2 composition of embodiment 46, wherein the AAV2 composition comprises 5-8×10¹³ vg/ml.
   48. The AAV2 composition of embodiment 47, wherein the AAV2 composition comprises 7-8×10¹³ vg/ml.
   49. The AAV2 composition of any one of embodiments 26-48, wherein the composition is stable during filtration/diafiltration up to 2, 3, 4, 5, 6, or 7 diavolumes.
   50. The AAV2 composition of any one of claims 26-49, wherein the composition is stable for at least one month, one year, or two years at 2-8° C.
   51. A method of producing an ultrafiltration/diafiltration (UFDF) AAV2 product, the method comprising filtering an isolated AAV2 composition using ultrafiltration/diafiltration (UFDF) to produce an AAV2 UFDF product, wherein the AAV2 composition comprises
AAV2 particles containing a transgene, and wherein the UFDF AAV2 product comprises a buffer comprising trehalose of greater than 5% w/v.
   52. The method of embodiment 51, wherein the buffer comprising trehalose is added before UFDF.
   53. The method of embodiment 51 or 52, wherein the buffer comprising trehalose is added after DF.
   54. The method of any one of embodiments 51-53, wherein the amount of particle aggregation is comparable before and after UFDF.
   55. The method of any one of embodiments 51-54, wherein the TMP was maintained during concentration between 6 and 8 psi.
   56. The method of any one of embodiments 51-55, further comprising adding a volume of the buffer equivalent to 1.7× the volume after diafiltration and depolarizing the product at a constant sheer rate of 500s-1 for 5 minutes.
   57. A method of producing an ultrafiltration/diafiltration (UFDF) AAV2 product, the method comprising filtering an isolated AAV2 composition using ultrafiltration/diafiltration (UFDF) to produce an AAV2 UFDF product, wherein the AAV2 composition comprises
AAV2 particles containing a transgene, and wherein the UFDF AAV2 product comprises a buffer comprising sucrose or glycerol of greater than 5% w/v.
   58. The method of embodiment 57, wherein the buffer comprising sucrose or glycerol is added before UFDF.
   59. The method of embodiment 57 or 58, wherein the buffer is added after DF.
   60. The method of any one of embodiments 57-59, wherein the amount of particle aggregation is comparable before and after UFDF.
   61. The method of any one of embodiments 57-60, wherein the TMP was maintained during concentration between 6 and 8 psi.
   62. The method of any one of embodiments 57-61, further comprising adding a volume of the buffer equivalent to 1.7× the volume after diafiltration and depolarizing the product at a constant sheer rate of 500s-1 for 5 minutes.
   63. An AAV2 product produced by the method of any one of embodiments 51-62.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

1. An adenovirus-associated virus type 2 (AAV2) composition comprising:
(a) AAV2 particles comprising a transgene; and
(b) a buffer comprising greater than 5% w/v of an aggregation-reducing additive;
wherein the AAV2 composition has less AAV2 particle aggregation than a control AAV2 composition in a buffer lacking the additive.

2. The AAV2 composition of claim 1, wherein the additive is trehalose.

3. The AAV2 composition of claim 1, wherein the buffer comprises histidine.

4. The AAV2 composition of claim 1, wherein the concentration of the additive in the buffer is 6-11%.

5. The AAV2 composition of claim 1, wherein the buffer does not comprise citrate.

6. The AAV2 composition of claim 1, wherein the buffer does not comprise potassium.

7. The AAV2 composition of claim 3, wherein the buffer comprises 15-25 mM histidine.

8. The AAV2 composition of claim 1, wherein the buffer further comprises a salt.

9. The AAV2 composition of claim 8, wherein the salt is sodium chloride.

10. The AAV2 composition of claim 1, wherein the buffer further comprises a surfactant.

11. The AAV2 composition of claim 10, wherein the surfactant is Poloxamer-188.

12. The AAV2 composition of claim 1, wherein the AAV2 composition has a pH of 7.3.

13. The AAV2 composition of claim 1, wherein the AAV2 composition is a chromatography product.

14. The AAV2 composition of claim 1, wherein aggregation is detected by dynamic light scattering or by size exclusion chromatography.

15. The AAV2 composition of claim 1, wherein the AAV2 composition comprises greater than 1×10¹³ vg/ml.

16. The AAV2 composition of claim 1, wherein the composition is stable during filtration/diafiltration up to 7 diavolumes.

17. The AAV2 composition of claim 1, wherein the composition is stable for at least one year at 2-8° C.

18. A method of producing an ultrafiltration/diafiltration (UFDF) AAV2 product, the method comprising filtering an isolated AAV2 composition using UFDF to produce an AAV2 UFDF product, wherein the AAV2 composition comprises AAV2 particles comprising a transgene, and wherein the UFDF AAV2 product comprises a buffer comprising greater than 5% w/v trehalose.

19. The method of claim 18, wherein the buffer comprising trehalose is added before UFDF.

20. The method of claim 18, wherein the buffer comprising trehalose is added after diafiltration.
